SEQUENCE LISTING (57 sequences)

SEQ ID NO: 1 - protein, 12 aa, Artificial
  Synthetic Peptide
CSFPKCPWVEGC

SEQ ID NO: 2 - protein, 11 aa, Artificial
  Synthetic Peptide
CRTFGCWWEGC

SEQ ID NO: 3 - protein, 15 aa, Artificial
  Synthetic Peptide
CLENCYYVPYYGYAC

SEQ ID NO: 4 - protein, 12 aa, Artificial
  Synthetic Peptide
  Xaa (5)..(5): Xaa is K(Ac)
CSFPXCPWVEGC

SEQ ID NO: 5 - protein, 12 aa, Artificial
  Synthetic Peptide
CYFPGCPWVENC

SEQ ID NO: 6 - protein, 12 aa, Artificial
  Synthetic Peptide
CQFPVCPWVEGC

SEQ ID NO: 7 - protein, 12 aa, Artificial
  Synthetic Peptide
CSFPMCPWVENC

SEQ ID NO: 8 - protein, 12 aa, Artificial
  Synthetic Peptide
CSFPKCPWVEKC

SEQ ID NO: 9 - protein, 12 aa, Artificial
  Synthetic Peptide
CSFPKCPWVEAC

SEQ ID NO: 10 - protein, 12 aa, Artificial
  Synthetic Peptide
  Xaa (8)..(8): Xaa is 2Nal
CSFPKCPXVEGC

SEQ ID NO: 11 - protein, 12 aa, Artificial
  Synthetic Peptide
  Xaa (8)..(8): Xaa is 1Nal
CSFPKCPXVEGC

SEQ ID NO: 12 - protein, 12 aa, Artificial
  Synthetic Peptide
  Xaa (5)..(5): Xaa is HArg
CSFPXCPWVEGC

SEQ ID NO: 13 - protein, 12 aa, Artificial
  Synthetic Peptide
CAFPKCPWVEGC

SEQ ID NO: 14 - protein, 12 aa, Artificial
  Synthetic Peptide
CSAPKCPWVEGC

SEQ ID NO: 15 - protein, 12 aa, Artificial
  Synthetic Peptide
CSFAKCPWVEGC

SEQ ID NO: 16 - protein, 12 aa, Artificial
  Synthetic Peptide
CSFPACPWVEGC

SEQ ID NO: 17 - protein, 12 aa, Artificial
  Synthetic Peptide
CSFPKCAWVEGC

SEQ ID NO: 18 - protein, 12 aa, Artificial
  Synthetic Peptide
CSFPKCPWVAGC

SEQ ID NO: 19 - protein, 12 aa, Artificial
  Synthetic Peptide
CSFPKCPWVEAC

SEQ ID NO: 20 - protein, 12 aa, Artificial
  Synthetic Peptide
CKFPVCPWVEYC

SEQ ID NO: 21 - protein, 12 aa, Artificial
  Synthetic Peptide
CHFPTCPWVEWC

SEQ ID NO: 22 - protein, 12 aa, Artificial
  Synthetic Peptide
CTFPACPWVEYC

SEQ ID NO: 23 - protein, 12 aa, Artificial
  Synthetic Peptide
CLFPGCPWVEHC

SEQ ID NO: 24 - protein, 12 aa, Artificial
  Synthetic Peptide
CFFPQCPWVEGC

SEQ ID NO: 25 - protein, 12 aa, Artificial
  Synthetic Peptide
CHFPVCPWVENC

SEQ ID NO: 26 - protein, 12 aa, Artificial
  Synthetic Peptide
CHFPKCPWVEGC

SEQ ID NO: 27 - protein, 12 aa, Artificial
  Synthetic Peptide
CEFPKCPWVEGC

SEQ ID NO: 28 - protein, 12 aa, Artificial
  Synthetic Peptide
CYFPKCPWVEGC

SEQ ID NO: 29 - protein, 12 aa, Artificial
  Synthetic Peptide
CSYPKCPWVEGC

SEQ ID NO: 30 - protein, 12 aa, Artificial
  Synthetic Peptide
CSFPKCPWVEHC

SEQ ID NO: 31 - protein, 12 aa, Artificial
  Synthetic Peptide
CTFPKCPWVEGC

SEQ ID NO: 32 - protein, 12 aa, Artificial
  Synthetic Peptide
CSFPKCPWVENC

SEQ ID NO: 33 - protein, 12 aa, Artificial
  Synthetic Peptide
CWFPNCPWVEGC

SEQ ID NO: 34 - protein, 12 aa, Artificial
  Synthetic Peptide
CYYPKCPWVEGC

SEQ ID NO: 35 - protein, 12 aa, Artificial
  Synthetic Peptide
CRFPKCPWVEGC

SEQ ID NO: 36 - protein, 12 aa, Artificial
  Synthetic Peptide
CSFPICPWVEGC

SEQ ID NO: 37 - protein, 12 aa, Artificial
  Synthetic Peptide
CVYPKCPWVEGC

SEQ ID NO: 38 - protein, 12 aa, Artificial
  Synthetic Peptide
CIYPKCPWVEGC

SEQ ID NO: 39 - protein, 12 aa, Artificial
  Synthetic Peptide
CSFAKCPWVEGC

SEQ ID NO: 40 - protein, 12 aa, Artificial
  Synthetic Peptide
CSFPACPWVEGC

SEQ ID NO: 41 - protein, 12 aa, Artificial
  Synthetic Peptide
CSFPTCPWVEGC

SEQ ID NO: 42 - protein, 12 aa, Artificial
  Synthetic Peptide
CVFPKCPWVEGC

SEQ ID NO: 43 - protein, 12 aa, Artificial
  Synthetic Peptide
CSFPHCPWVEGC

SEQ ID NO: 44 - protein, 12 aa, Artificial
  Synthetic Peptide
CHYPKCPWVEGC

SEQ ID NO: 45 - protein, 12 aa, Artificial
  Synthetic Peptide
CEYPKCPWVEGC

SEQ ID NO: 46 - protein, 12 aa, Artificial
  Synthetic Peptide
CSFPLCPWVEGC

SEQ ID NO: 47 - protein, 12 aa, Artificial
  Synthetic Peptide
CAFPHCPWVEFC

SEQ ID NO: 48 - protein, 12 aa, Artificial
  Synthetic Peptide
CQFPGCPWVEFC

SEQ ID NO: 49 - protein, 12 aa, Artificial
  Synthetic Peptide
CIFPKCPWVEGC

SEQ ID NO: 50 - protein, 12 aa, Artificial
  Synthetic Peptide
CSFPSCPWVEHC

SEQ ID NO: 51 - protein, 12 aa, Artificial
  Synthetic Peptide
CWFPGCPWVEHC

SEQ ID NO: 52 - protein, 12 aa, Artificial
  Synthetic Peptide
CRFPDCPWVEHC

SEQ ID NO: 53 - protein, 12 aa, Artificial
  Synthetic Peptide
CHFPTCPWVEYC

SEQ ID NO: 54 - protein, 12 aa, Artificial
  Synthetic Peptide
CVFPACPWVEYC

SEQ ID NO: 55 - protein, 12 aa, Artificial
  Synthetic Peptide
CNFPECPWVEHC

SEQ ID NO: 56 - protein, 12 aa, Artificial
  Synthetic Peptide
CRFPNCPWVEHC

SEQ ID NO: 57 - protein, 12 aa, Artificial
  Synthetic Peptide
CVFPTCPWVEHC

(19) United States
(12) Patent Application Publication
(10) Pub. No.: US 2023/0106511 A1
(43) Pub. Date: Apr. 6, 2023

(54) PBP3 BINDING BICYCLIC PEPTIDE LIGANDS

(71) Applicant: BicycleTx Limited, Cambridge (GB)

(72) Inventors: Matthew BALMFORTH, Cambridge (GB); Paul BESWICK, Cambridge (GB); Liuhong CHEN, Cambridge (GB); Mike DAWSON, Cambridge (GB); Rachel DODS, Cambridge (GB); Catherine ROWLAND, Cambridge (GB); Michael SKYNNER, Cambridge (GB); Katerine VAN RIETSCHOTEN, Cambridge (GB)

(21) Appl. No.: 17/905,010

(22) PCT Filed: Feb. 26, 2021
(86) PCT No.: PCT/GB2021/050491

§ 371 Date: Aug. 25, 2022

(30) Foreign Application Priority Data
Feb. 26, 2020 (GB) 2002706.6

(51) Int. Cl. C07K 7/08; A61P 31/04

(52) U.S. Cl. CPC C07K 7/08; A61P 31/04; A61K 38/00

(57) ABSTRACT

The present invention relates to polypeptides which are covalently bound to molecular scaffolds such that two or more peptide loops are subtended between attachment points to the scaffold. In particular, the invention describes peptides which are high affinity binders of penicillin-binding proteins (PBPs) more particularly PBP3. The invention also includes pharmaceutical compositions comprising said peptide ligands and to the use of said peptide ligands in suppressing or treating a disease or disorder mediated by bacterial infection or for providing prophylaxis to a subject at risk of infection.

### FIELD OF THE INVENTION

The present invention relates to polypeptides which are covalently bound to molecular scaffolds such that two or more peptide loops are subtended between attachment points to the scaffold. In particular, the invention describes peptides which are high affinity binders of penicillin-binding proteins (PBPs) more particularly PBP3. The invention also includes pharmaceutical compositions comprising said peptide ligands and to the use of said peptide ligands in suppressing or treating a disease or disorder mediated by bacterial infection or for providing prophylaxis to a subject at risk of infection.

### BACKGROUND OF THE INVENTION

Cyclic peptides are able to bind with high affinity and target specificity to protein targets and hence are an attractive molecule class for the development of therapeutics. In fact, several cyclic peptides are already successfully used in the clinic, as for example the antibacterial peptide vancomycin, the immunosuppressant drug cyclosporine or the anti-cancer drug octreotide (Driggers et al. (2008), Nat Rev Drug Discov 7 (7), 608-24). Good binding properties result from a relatively large interaction surface formed between the peptide and the target as well as the reduced conformational flexibility of the cyclic structures. Typically, macrocycles bind to surfaces of several hundred square angstrom, as for example the cyclic peptide CXCR4 antagonist CVX15 (400 Å²; Wu et al. (2007), Science 330, 1066-71), a cyclic peptide with the Arg-Gly-Asp motif binding to integrin αVb3 (355 Å²) (Xiong et al. (2002), Science 296 (5565), 151-5) or the cyclic peptide inhibitor upain-1 binding to urokinase-type plasminogen activator (603 Å²; Zhao et al. (2007), J Struct Biol 160 (1), 1-10).

Due to their cyclic configuration, peptide macrocycles are less flexible than linear peptides, leading to a smaller loss of entropy upon binding to targets and resulting in a higher binding affinity. The reduced flexibility also leads to locking target-specific conformations, increasing binding specificity compared to linear peptides. This effect has been exemplified by a potent and selective inhibitor of matrix metalloproteinase 8 (MMP-8) which lost its selectivity over other MMPs when its ring was opened (Cherney et al. (1998), J Med Chem 41 (11), 1749-51). The favorable binding properties achieved through macrocyclization are even more pronounced in multicyclic peptides having more than one peptide ring as for example in vancomycin, nisin and actinomycin.

Different research teams have previously tethered polypeptides with cysteine residues to a synthetic molecular structure (Kemp and McNamara (1985), J. Org. Chem; Timmerman et al. (2005), ChemBioChem). Meloen and co-workers had used tris(bromomethyl)benzene and related molecules for rapid and quantitative cyclisation of multiple peptide loops onto synthetic scaffolds for structural mimicry of protein surfaces (Timmerman et al. (2005), ChemBioChem). Methods for the generation of candidate drug compounds wherein said compounds are generated by linking cysteine containing polypeptides to a molecular scaffold as for example tris(bromomethyl)benzene are disclosed in WO 2004/077062 and WO 2006/078161.

Phage display-based combinatorial approaches have been developed to generate and screen large libraries of bicyclic peptides to targets of interest (Heinis et al. (2009), Nat Chem Biol 5 (7), 502-7 and WO 2009/098450). Briefly, combinatorial libraries of linear peptides containing three cysteine residues and two regions of six random amino acids (Cys-(Xaa)₆-Cys-(Xaa)₆-Cys) were displayed on phage and cyclised by covalently linking the cysteine side chains to a small molecule scaffold.

### SUMMARY OF THE INVENTION

According to a first aspect of the invention, there is provided a peptide ligand capable of binding to a penicillin-binding protein 3 (PBP3) comprising a polypeptide which comprises at least three cysteine residues, separated by at least two loop sequences, and a molecular scaffold which forms covalent bonds with the cysteine residues of the polypeptide such that at least two polypeptide loops are formed on the molecular scaffold.

According to a further aspect of the invention, there is provided a pharmaceutical composition comprising a peptide ligand as defined herein in combination with one or more pharmaceutically acceptable excipients.

According to a further aspect of the invention, there is provided a peptide ligand as defined herein for use in suppressing or treating a disease or disorder mediated by bacterial infection or for providing prophylaxis to a subject at risk of infection.

### DETAILED DESCRIPTION OF THE INVENTION

In one embodiment, said loop sequences comprise 3, 4, 5 or 9 amino acids.

In a further embodiment, said loop sequences comprise three cysteine residues separated by two loop sequences both of which consist of 4 amino acids.

In a further embodiment, said loop sequences comprise three cysteine residues separated by two loop sequences one of which consists of 4 amino acids and the other of which consists of 5 amino acids.

In a further embodiment, said loop sequences comprise three cysteine residues separated by two loop sequences one of which consists of 3 amino acids and the other of which consists of 9 amino acids.

References herein to PBP include a “penicillin-binding protein” which may be present in any bacterial species. In one embodiment, the PBP is a PBP which is present within one or more pathogenic bacterial species. In a further embodiment, the one or more pathogenic bacterial species is selected from any of: Acinetobacter baumannii, Bacillus anthracis, Bordetella pertussis, Borrelia burgdorferi, Brucella abortus, Brucella canis, Brucella melitensis, Brucella suis, Campylobacter jejuni, Chlamydia pneumonia, Chlamydia trachomatis, Chlamydophila psittaci, Clostridium botulinum, Clostridium difficile, Clostridium perfringens, Clostrium tetani, Corynebacterium diphtheriae, Echinococcus, Enterococcus faecalis, Enterococcus faecium, Escherichia coli (such as Enterotoxigenic E. coli, Enteropathogenic E. coli, Enterohemorragic E. coli or Enteroaggregative E. coli), Francisella tularensis, Haemophilus influenzae, Helicobacter pylori, Klebsiella pneumoniae, Legionella pneumophila, Leptospira interrogans, Listeria monocytogenes, Mycobacterium leprae, Mycobacterium tuberculosis, Mycobacterium ulcerans, Mycoplasma pneumonia, Neisseria gonorrhoeae, Neisseria meningitides, Pneumococcus, Pseudomonas aeruginosa, Rickettsia rickettsia, Salmonella such as, Salmonella bongori, Salmonella enterica, Salmonella subterranean, Salmonella typhi or Salmonella typhimurium, Shigella (such as Shigella sonnei or Shigella dysenteriae), Staphylococcus aureus (such as MRSA), Staphylococcus epidermidis, Staphylococcus saprophyticus, Streptococcus agalactiae, Streptococcus pneumoniae, Streptococcus pyogenes, Treponema pallidum, Vibrio cholerae or Yersinia pestis.

In one embodiment, the PBP3 is a PBP3 which is present within E. coli.

In an alternative embodiment, the PBP3 is a PBP3 which is present within P. aeruginosa. In a further embodiment, the PBP3 present within P. aeruginosa is selected from PBP3 and PBP3a. In a yet further embodiment, the PBP3 present within P. aeruginosa is PBP3.

In an alternative embodiment, the PBP3 is a PBP3 which is present within A. baumannii

In one embodiment, the PBP3 is required for cell division, such as Ftsl. In a further embodiment, the Ftsl is present in E. coli, A. baumannii or P. aeruginosa and is known as PBP3. Thus, according to certain embodiments of the present invention, PBP3 is Ftsl.

In one embodiment, the PBP3 is E. coli PBP3 and the peptide ligand comprises an amino acid sequence selected from:

(SEQ ID NO: 1)
CᵢSFPKCᵢᵢPWVEGCᵢᵢᵢ
(herein referred to as BCY13949);
(SEQ ID NO: 2)
CᵢRTFGCᵢᵢWWEGCᵢᵢᵢ;
(SEQ ID NO: 3)
CᵢLENCᵢᵢYYVPYYGYACᵢᵢᵢ;
(SEQ ID NO: 4)
CᵢSFP[K(Ac)]CᵢᵢPWVEGCᵢᵢᵢ;
(SEQ ID NO: 5)
CᵢYFPGCᵢᵢPWVENCᵢᵢᵢ;
(SEQ ID NO: 6)
CᵢQFPVCᵢᵢPWVEGCᵢᵢᵢ;
(SEQ ID NO: 7)
CᵢSFPMCᵢᵢPWVENCᵢᵢᵢ;
(SEQ ID NO: 8)
CᵢSFPKCᵢᵢPWVE[dK]Cᵢᵢᵢ;
(SEQ ID NO: 9)
CᵢSFPKCᵢᵢPWVE[dA]Cᵢᵢᵢ;
(SEQ ID NO: 10)
CᵢSFPKCᵢᵢP[2Nal]VEGCᵢᵢᵢ;
(SEQ ID NO: 11)
CᵢSFPKCᵢᵢP[1Nal]VEGCᵢᵢᵢ;
(SEQ ID NO: 12)
CᵢSFP[HArg]CᵢᵢPWVEGCᵢᵢᵢ;
(SEQ ID NO: 13)
CᵢAFPKCᵢᵢPWVEGCᵢᵢᵢ;
(SEQ ID NO: 14)
CᵢSAPKCᵢᵢPWVEGCᵢᵢᵢ;
(SEQ ID NO: 15)
CᵢSFAKCᵢᵢPWVEGCᵢᵢᵢ;
(SEQ ID NO: 16)
CᵢSFPACᵢᵢPWVEGCᵢᵢᵢ;
(SEQ ID NO: 17)
CᵢSFPKCᵢᵢAWVEGCᵢᵢᵢ;
(SEQ ID NO: 18)
CᵢSFPKCᵢᵢPWVAGCᵢᵢᵢ;
(SEQ ID NO: 19)
CᵢSFPKCᵢᵢPWVEACᵢᵢᵢ;
(SEQ ID NO: 20)
CᵢKFPVCᵢᵢPWVEYCᵢᵢᵢ;
(SEQ ID NO: 21)
CᵢHFPTCᵢᵢPWVEWCᵢᵢᵢ;
(SEQ ID NO: 22)
CᵢTFPACᵢᵢPWVEYCᵢᵢᵢ;
(SEQ ID NO: 23)
CᵢLFPGCᵢᵢPWVEHCᵢᵢᵢ;
(SEQ ID NO: 24)
CᵢFFPQCᵢᵢPWVEGCᵢᵢᵢ;
(SEQ ID NO: 25)
CᵢHFPVCᵢᵢPWVENCᵢᵢᵢ;
(SEQ ID NO: 26)
CᵢHFPKCᵢᵢPWVEGCᵢᵢᵢ;
(SEQ ID NO: 27)
CᵢEFPKCᵢᵢPWVEGCᵢᵢᵢ;
(SEQ ID NO: 28)
CᵢYFPKCᵢᵢPWVEGCᵢᵢᵢ;
(SEQ ID NO: 29)
CᵢSYPKCᵢᵢPWVEGCᵢᵢᵢ;
(SEQ ID NO: 30)
CᵢSFPKCᵢᵢPWVEHCᵢᵢᵢ;
(SEQ ID NO: 31)
CᵢTFPKCᵢᵢPWVEGCᵢᵢᵢ;
(SEQ ID NO: 32)
CᵢSFPKCᵢᵢPWVENCᵢᵢᵢ;
(SEQ ID NO: 33)
CᵢWFPNCᵢᵢPWVEGCᵢᵢᵢ;
(SEQ ID NO: 34)
CᵢYYPKCᵢᵢPWVEGCᵢᵢᵢ;
(SEQ ID NO: 35)
CᵢRFPKCᵢᵢPWVEGCᵢᵢᵢ:
(SEQ ID NO: 36)
CᵢSFPICᵢᵢPWVEGCᵢᵢᵢ;
(SEQ ID NO: 37)
CᵢVYPKCᵢᵢPWVEGCᵢᵢᵢ;
(SEQ ID NO: 38)
CᵢIYPKCᵢᵢPWVEGCᵢᵢᵢ;
(SEQ ID NO: 39)
CᵢSF[dA]KCᵢᵢPWVEGCᵢᵢᵢ;
(SEQ ID NO: 40)
CᵢSFP[dA]CᵢᵢPWVEGCᵢᵢᵢ;
(SEQ ID NO: 41)
CᵢSFPTCᵢᵢPWVEGCᵢᵢᵢ;
(SEQ ID NO: 42)
CᵢVFPKCᵢᵢPWVEGCᵢᵢᵢ;
(SEQ ID NO: 43)
CᵢSFPHCᵢᵢPWVEGCᵢᵢᵢ;
(SEQ ID NO: 44)
CᵢHYPKCᵢᵢPWVEGCᵢᵢᵢ;
(SEQ ID NO: 45)
CᵢEYPKCᵢᵢPWVEGCᵢᵢᵢ;
(SEQ ID NO: 46)
CᵢSFPLCᵢᵢPWVEGCᵢᵢᵢ;
(SEQ ID NO: 47)
CᵢAFPHCᵢᵢPWVEFCᵢᵢᵢ;
(SEQ ID NO: 48)
CᵢQFPGCᵢᵢPWVEFCᵢᵢᵢ;
(SEQ ID NO: 49)
CᵢIFPKCᵢᵢPWVEGCᵢᵢᵢ;
(SEQ ID NO: 50)
CᵢSFPSCᵢᵢPWVEHCᵢᵢᵢ;
(SEQ ID NO: 51)
CᵢWFPGCᵢᵢPWVEHCᵢᵢᵢ;
(SEQ ID NO: 52)
CᵢRFPDCᵢᵢPWVEHCᵢᵢᵢ;
(SEQ ID NO: 53)
CᵢHFPTCᵢᵢPWVEYCᵢᵢᵢ;
(SEQ ID NO: 54)
CᵢVFPACᵢᵢPWVEYCᵢᵢᵢ;
(SEQ ID NO: 55)
CᵢNFPECᵢᵢPWVEHCᵢᵢᵢ;
(SEQ ID NO: 56)
CᵢRFPNCᵢᵢPWVEHCᵢᵢᵢ;
and
(SEQ ID NO: 57)
CᵢVFPTCᵢᵢPWVEHCᵢᵢᵢ;

wherein HArg represent homoarginine, 1Nal represents 1-naphthylalanine, 2Nal represents 2-naphthylalanine and Cᵢ, Cᵢᵢ and Cᵢᵢᵢ represent first, second and third cysteine residues or a pharmaceutically acceptable salt thereof.

In a further embodiment, the PBP3 is E. coli PBP3 and the peptide ligand additionally comprises N- and/or C-terminal additions and comprises an amino acid sequence selected from:

Ac-(SEQ ID NO: 1)
(herein referred to as BCY12742);
A-(SEQ ID NO: 1)-A-[Sar₆]-[KFI]
(herein referred to as BCY12806);
[FI]-G-[Sar₅]-A-(SEQ ID NO: 1)-A
(herein referred to as BCY12820);
SGH-(SEQ ID NO: 1)-A
(herein referred to as BCY13761);
Ac-(SEQ ID NO: 2)
(herein referred to as BCY12744);
[FI]-G-[Sar₅]-A-(SEQ ID NO: 2)-A
(herein referred to as BCY12822);
A-(SEQ ID NO: 2)-A-[Sar₆]-[KFI]
(herein referred to as BCY12825);
A-(SEQ ID NO: 3)-A
(herein referred to as BCY12123);
Ac-(SEQ ID NO: 4);
(herein referred to as BCY12834);
A-(SEQ ID NO: 5)-A
(herein referred to as BCY13981);
A-(SEQ ID NO: 6)-A
(herein referred to as BCY13226);
A-(SEQ ID NO: 7)-A
(herein referred to as BCY13227);
A-(SEQ ID NO: 8)-A
(herein referred to as BCY13419);
A-(SEQ ID NO: 9)-A
(herein referred to as BCY13420);
A-(SEQ ID NO: 10)-A
(herein referred to as BCY13421);
A-(SEQ ID NO: 11)-A
(herein referred to as BCY13422);
A-(SEQ ID NO: 12)-A
(herein referred to as BCY13424);
A-(SEQ ID NO: 13)-A
(herein referred to as BCY13656);
A-(SEQ ID NO: 14)-A
(herein referred to as BCY13657);
A-(SEQ ID NO: 15)-A
(herein referred to as BCY13658);
A-(SEQ ID NO: 16)-A
(herein referred to as BCY13659);
A-(SEQ ID NO: 17)-A
(herein referred to as BCY13660);
A-(SEQ ID NO: 18)-A
(herein referred to as BCY13663);
A-(SEQ ID NO: 19)-A
(herein referred to as BCY13664);
A-(SEQ ID NO: 20)-A
(herein referred to as BCY13747);
A-(SEQ ID NO: 21)-A
(herein referred to as BCY13748);
A-(SEQ ID NO: 22)-A
(herein referred to as BCY13749);
A-(SEQ ID NO: 23)-A
(herein referred to as BCY13750);
A-(SEQ ID NO: 24)-A
(herein referred to as BCY13751);
A-(SEQ ID NO: 25)-A
(herein referred to as BCY13753);
A-(SEQ ID NO: 26)-A
(herein referred to as BCY13754);
EDR-(SEQ ID NO: 26)-A
(herein referred to as BCY13760);
A-(SEQ ID NO: 27)-A
(herein referred to as BCY13755);
A-(SEQ ID NO: 28)-A
(herein referred to as BCY13756);
A-(SEQ ID NO: 29)-A
(herein referred to as BCY13757);
A-(SEQ ID NO: 30)-A
(herein referred to as BCY13758);
A-(SEQ ID NO: 30)-SSI
(herein referred to as BCY13762);
ESW-(SEQ ID NO: 31)-A
(herein referred to as BCY13759);
A-(SEQ ID NO: 31)-A
(herein referred to as BCY13963);
A-(SEQ ID NO: 32)-KVD
(herein referred to as BCY13763);
A-(SEQ ID NO: 33)-A
(herein referred to as BCY13764);
A-(SEQ ID NO: 34)-A
(herein referred to as BCY13765);
A-(SEQ ID NO: 35)-A
(herein referred to as BCY13766);
A-(SEQ ID NO: 36)-A
(herein referred to as BCY13767);
A-(SEQ ID NO: 37)-A
(herein referred to as BCY13768);
A-(SEQ ID NO: 38)-A
(herein referred to as BCY13769);
A-(SEQ ID NO: 39)-A
(herein referred to as BCY13952);
A-(SEQ ID NO: 40)-A
(herein referred to as BCY13953);
A-(SEQ ID NO: 41)-A
(herein referred to as BCY13961);
A-(SEQ ID NO: 42)-A
(herein referred to as BCY13962);
A-(SEQ ID NO: 43)-A
(herein referred to as BCY13965);
A-(SEQ ID NO: 44)-A
(herein referred to as BCY13966);
A-(SEQ ID NO: 45)-A
(herein referred to as BCY13967);
A-(SEQ ID NO: 46)-A
(herein referred to as BCY13968);
A-(SEQ ID NO: 47)-A
(herein referred to as BCY13969);
A-(SEQ ID NO: 48)-A
(herein referred to as BCY13970);
A-(SEQ ID NO: 49)-A
(herein referred to as BCY13971);
A-(SEQ ID NO: 50)-A
(herein referred to as BCY13972);
A-(SEQ ID NO: 51)-A
(herein referred to as BCY13973);
A-(SEQ ID NO: 52)-A
(herein referred to as BCY13975);
A-(SEQ ID NO: 53)-A
(herein referred to as BCY13976);
A-(SEQ ID NO: 54)-A
(herein referred to as BCY13977);
A-(SEQ ID NO: 55)-A
(herein referred to as BCY13978);
A-(SEQ ID NO: 56)-A
(herein referred to as BCY13979);
and
A-(SEQ ID NO: 57)-A
(herein referred to as BCY13980).

Unless defined otherwise, all technical and scientific terms used herein have the same meaning as commonly understood by those of ordinary skill in the art, such as in the arts of peptide chemistry, cell culture and phage display, nucleic acid chemistry and biochemistry. Standard techniques are used for molecular biology, genetic and biochemical methods (see Sambrook et al., Molecular Cloning: A Laboratory Manual, 3rd ed., 2001, Cold Spring Harbor Laboratory Press, Cold Spring Harbor, N.Y.; Ausubel et al., Short Protocols in Molecular Biology (1999) 4^{th} ed., John Wiley & Sons, Inc.), which are incorporated herein by reference.

### Nomenclature

### Numbering

When referring to amino acid residue positions within peptides of the invention, cysteine residues (Cᵢ, Cᵢᵢ and Cᵢᵢᵢ) are omitted from the numbering as they are invariant, therefore, the numbering of amino acid residues within peptides of the invention is referred to as below:

(SEQ ID NO: 1)
CᵢS₁-F₂-P₃-K₄-Cᵢᵢ-P₅-W₆-V₇-E₈-G₉-Cᵢᵢᵢ.

For the purpose of this description, all bicyclic peptides are assumed to be cyclised with 1,1′,1″-(1,3,5-triazinane-1,3,5-triyl)triprop-2-en-1-one (TATA) and yielding a tri-substituted structure. Cyclisation with TATA occurs Cᵢ, Cᵢᵢ and Cᵢᵢᵢ.

### Molecular Format

N- or C-terminal extensions to the bicycle core sequence are added to the left or right side of the sequence, separated by a hyphen. For example, an N-terminal βAla-Sar10-Ala tail would be denoted as:

(SEQ ID NO: X)
βAla-Sar₁₀-A-.

### Inversed Peptide Sequences

In light of the disclosure in Nair et al (2003) J Immunol 170 (3), 1362-1373, it is envisaged that the peptide sequences disclosed herein would also find utility in their retro-inverso form. For example, the sequence is reversed (i.e. N-terminus becomes C-terminus and vice versa) and their stereochemistry is likewise also reversed (i.e. D-amino acids become L-amino acids and vice versa).

### Peptide Ligands

A peptide ligand, as referred to herein, refers to a peptide covalently bound to a molecular scaffold. Typically, such peptides comprise two or more reactive groups (i.e. cysteine residues) which are capable of forming covalent bonds to the scaffold, and a sequence subtended between said reactive groups which is referred to as the loop sequence, since it forms a loop when the peptide is bound to the scaffold. In the present case, the peptides comprise at least three cysteine residues (referred to herein as Cᵢ, Cᵢᵢ and Cᵢᵢᵢ), and form at least two loops on the scaffold.

### Advantages of the Peptide Ligands

Certain bicyclic peptides of the present invention have a number of advantageous properties which enable them to be considered as suitable drug-like molecules for injection, inhalation, nasal, ocular, oral or topical administration. Such advantageous properties include:
Species cross-reactivity. Certain ligands demonstrate cross-reactivity across PBPs from different bacterial species and hence are able to treat infections caused by multiple species of bacteria. Other ligands may be highly specific for the PBPs of certain bacterial species which may be advantageous for treating an infection without collateral damage to the beneficial flora of the patient;
Protease stability. Bicyclic peptide ligands should ideally demonstrate stability to plasma proteases, epithelial (“membrane-anchored”) proteases, gastric and intestinal proteases, lung surface proteases, intracellular proteases and the like. Protease stability should be maintained between different species such that a bicycle lead candidate can be developed in animal models as well as administered with confidence to humans;
Desirable solubility profile. This is a function of the proportion of charged and hydrophilic versus hydrophobic residues and intra/inter-molecular H-bonding, which is important for formulation and absorption purposes;
An optimal plasma half-life in the circulation. Depending upon the clinical indication and treatment regimen, it may be required to develop a bicyclic peptide for short exposure in an acute illness management setting, or develop a bicyclic peptide with enhanced retention in the circulation, and is therefore optimal for the management of more chronic disease states. Other factors driving the desirable plasma half-life are requirements of sustained exposure for maximal therapeutic efficiency versus the accompanying toxicology due to sustained exposure of the agent; and
Selectivity. Certain peptide ligands of the invention demonstrate selectivity for a particular PBP3 isoform and certain other peptide ligands of the invention may inhibit more than one PBP isoform.

### Pharmaceutically Acceptable Salts

It will be appreciated that salt forms are within the scope of this invention, and references to peptide ligands include the salt forms of said ligands.

The salts of the present invention can be synthesized from the parent compound that contains a basic or acidic moiety by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free acid or base forms of these compounds with the appropriate base or acid in water or in an organic solvent, or in a mixture of the two.

Acid addition salts (mono- or di-salts) may be formed with a wide variety of acids, both inorganic and organic. Examples of acid addition salts include mono- or di-salts formed with an acid selected from the group consisting of acetic, 2,2-dichloroacetic, adipic, alginic, ascorbic (e.g. L-ascorbic), L-aspartic, benzenesulfonic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrohalic acids (e.g. hydrobromic, hydrochloric, hydriodic), isethionic, lactic (e.g. (+)-L-lactic, (±)-DL-lactic), lactobionic, maleic, malic, (−)-L-malic, malonic, (±)-DL-mandelic, methanesulfonic, naphthalene-2-sulfonic, naphthalene-1,5-disulfonic, 1-hydroxy-2-naphthoic, nicotinic, nitric, oleic, orotic, oxalic, palmitic, pamoic, phosphoric, propionic, pyruvic, L-pyroglutamic, salicylic, 4-amino-salicylic, sebacic, stearic, succinic, sulfuric, tannic, (+)-L-tartaric, thiocyanic, ρ-toluenesulfonic, undecylenic and valeric acids, as well as acylated amino acids and cation exchange resins.

One particular group of salts consists of salts formed from acetic, hydrochloric, hydriodic, phosphoric, nitric, sulfuric, citric, lactic, succinic, maleic, malic, isethionic, fumaric, benzenesulfonic, toluenesulfonic, sulfuric, methanesulfonic (mesylate), ethanesulfonic, naphthalenesulfonic, valeric, propanoic, butanoic, malonic, glucuronic and lactobionic acids. One particular salt is the hydrochloride salt. Another particular salt is the acetate salt.

If the compound is anionic, or has a functional group which may be anionic (e.g., —COOH may be —COO⁻), then a salt may be formed with an organic or inorganic base, generating a suitable cation. Examples of suitable inorganic cations include, but are not limited to, alkali metal ions such as Li⁺, Na⁺ and K⁺, alkaline earth metal cations such as Ca²⁺ and Mg²⁺, and other cations such as Al³⁺ or Zn⁺. Examples of suitable organic cations include, but are not limited to, ammonium ion (i.e., NH₄⁺) and substituted ammonium ions (e.g., NH₃R⁺, NH₂R₂₊, NHR₃⁺, NR₄⁺). Examples of some suitable substituted ammonium ions are those derived from: methylamine, ethylamine, diethylamine, propylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the peptides of the invention contain an amine function, these may form quaternary ammonium salts, for example by reaction with an alkylating agent according to methods well known to the skilled person. Such quaternary ammonium compounds are within the scope of the peptides of the invention.

### Modified Derivatives

It will be appreciated that modified derivatives of the peptide ligands as defined herein are within the scope of the present invention. Examples of such suitable modified derivatives include one or more modifications selected from: N-terminal and/or C-terminal modifications; replacement of one or more amino acid residues with one or more non-natural amino acid residues (such as replacement of one or more polar amino acid residues with one or more isosteric or isoelectronic amino acids; replacement of one or more non-polar amino acid residues with other non-natural isosteric or isoelectronic amino acids); addition of a spacer group; replacement of one or more oxidation sensitive amino acid residues with one or more oxidation resistant amino acid residues; replacement of one or more amino acid residues with an alanine, replacement of one or more L-amino acid residues with one or more D-amino acid residues; N-alkylation of one or more amide bonds within the bicyclic peptide ligand; replacement of one or more peptide bonds with a surrogate bond; peptide backbone length modification; substitution of the hydrogen on the alpha-carbon of one or more amino acid residues with another chemical group, modification of amino acids such as cysteine, lysine, glutamate/aspartate and tyrosine with suitable amine, thiol, carboxylic acid and phenol-reactive reagents so as to functionalise said amino acids, and introduction or replacement of amino acids that introduce orthogonal reactivities that are suitable for functionalisation, for example azide or alkyne-group bearing amino acids that allow functionalisation with alkyne or azide-bearing moieties, respectively.

In one embodiment, the modified derivative comprises an N-terminal and/or C-terminal modification. In a further embodiment, wherein the modified derivative comprises an N-terminal modification using suitable amino-reactive chemistry, and/or C-terminal modification using suitable carboxy-reactive chemistry. In a further embodiment, said N-terminal or C-terminal modification comprises addition of an effector group, including but not limited to a cytotoxic agent, a radiochelator or a chromophore.

In a further embodiment, the modified derivative comprises an N-terminal modification. In a further embodiment, the N-terminal modification comprises an N-terminal acetyl group. In this embodiment, the N-terminal cysteine group (the group referred to herein as C₁) is capped with acetic anhydride or other appropriate reagents during peptide synthesis leading to a molecule which is N-terminally acetylated. This embodiment provides the advantage of removing a potential recognition point for aminopeptidases and avoids the potential for degradation of the bicyclic peptide.

In an alternative embodiment, the N-terminal modification comprises the addition of a molecular spacer group which facilitates the conjugation of effector groups and retention of potency of the bicyclic peptide to its target.

In a further embodiment, the modified derivative comprises a C-terminal modification. In a further embodiment, the C-terminal modification comprises an amide group. In this embodiment, the C-terminal cysteine group (the group referred to herein as Cₕi) is synthesized as an amide during peptide synthesis leading to a molecule which is C-terminally amidated. This embodiment provides the advantage of removing a potential recognition point for carboxypeptidase and reduces the potential for proteolytic degradation of the bicyclic peptide.

In one embodiment, the modified derivative comprises replacement of one or more amino acid residues with one or more non-natural amino acid residues. In this embodiment, non-natural amino acids may be selected having isosteric/isoelectronic side chains which are neither recognised by degradative proteases nor have any adverse effect upon target potency.

Alternatively, non-natural amino acids may be used having constrained amino acid side chains, such that proteolytic hydrolysis of the nearby peptide bond is conformationally and sterically impeded. In particular, these concern proline analogues, bulky sidechains, Cα-disubstituted derivatives (for example, aminoisobutyric acid, Aib), and cyclo amino acids, a simple derivative being amino-cyclopropylcarboxylic acid.

In one embodiment, the modified derivative comprises the addition of a spacer group. In a further embodiment, the modified derivative comprises the addition of a spacer group to the N-terminal cysteine (Cᵢ) and/or the C-terminal cysteine (Cᵢᵢᵢ).

In one embodiment, the modified derivative comprises replacement of one or more oxidation sensitive amino acid residues with one or more oxidation resistant amino acid residues.

In one embodiment, the modified derivative comprises replacement of one or more charged amino acid residues with one or more hydrophobic amino acid residues. In an alternative embodiment, the modified derivative comprises replacement of one or more hydrophobic amino acid residues with one or more charged amino acid residues. The correct balance of charged versus hydrophobic amino acid residues is an important characteristic of the bicyclic peptide ligands. For example, hydrophobic amino acid residues influence the degree of plasma protein binding and thus the concentration of the free available fraction in plasma, while charged amino acid residues (in particular arginine) may influence the interaction of the peptide with the phospholipid membranes on cell surfaces. The two in combination may influence half-life, volume of distribution and exposure of the peptide drug, and can be tailored according to the clinical endpoint. In addition, the correct combination and number of charged versus hydrophobic amino acid residues may reduce irritation at the injection site (if the peptide drug has been administered subcutaneously).

In one embodiment, the modified derivative comprises replacement of one or more L-amino acid residues with one or more D-amino acid residues. This embodiment is believed to increase proteolytic stability by steric hindrance and by a propensity of D-amino acids to stabilise β-turn conformations (Tugyi et al (2005) PNAS, 102 (2), 413-418).

In one embodiment, the modified derivative comprises removal of any amino acid residues and substitution with alanines. This embodiment provides the advantage of removing potential proteolytic attack site(s).

It should be noted that each of the above mentioned modifications serve to deliberately improve the potency or stability of the peptide. Further potency improvements based on modifications may be achieved through the following mechanisms:
Incorporating hydrophobic moieties that exploit the hydrophobic effect and lead to lower off rates, such that higher affinities are achieved;
Incorporating charged groups that exploit long-range ionic interactions, leading to faster on rates and to higher affinities (see for example Schreiber et al, Rapid, electrostatically assisted association of proteins (1996), Nature Struct. Biol. 3, 427-31); and
Incorporating additional constraint into the peptide, by for example constraining side chains of amino acids correctly such that loss in entropy is minimal upon target binding, constraining the torsional angles of the backbone such that loss in entropy is minimal upon target binding and introducing additional cyclisations in the molecule for identical reasons.

(for reviews see Gentilucci et al, Curr. Pharmaceutical Design, (2010), 16, 3185-203, and Nestor et al, Curr. Medicinal Chem (2009), 16, 4399-418).

### Isotopic Variations

The present invention includes all pharmaceutically acceptable (radio)isotope-labeled peptide ligands of the invention, wherein one or more atoms are replaced by atoms having the same atomic number, but an atomic mass or mass number different from the atomic mass or mass number usually found in nature, and peptide ligands of the invention, wherein metal chelating groups are attached (termed “effector”) that are capable of holding relevant (radio)isotopes, and peptide ligands of the invention, wherein certain functional groups are covalently replaced with relevant (radio)isotopes or isotopically labelled functional groups.

Examples of isotopes suitable for inclusion in the peptide ligands of the invention comprise isotopes of hydrogen, such as ²H (D) and ³H (T), carbon, such as ¹¹C, ¹³C and ¹⁴C, chlorine, such as ³⁶Cl, fluorine, such as ¹⁸F, iodine, such as 123I, 125I and ¹³¹I, nitrogen, such as ¹³N and ¹⁵N, oxygen, such as ¹⁵O, ¹⁷O and ¹⁸O, phosphorus, such as ³²P, sulfur, such as ³⁵S, copper, such as ⁶⁴Cu, gallium, such as ⁶⁷Ga or ⁶⁸Ga, yttrium, such as ⁹⁰Y and lutetium, such as ¹⁷⁷Lu, and Bismuth, such as ²¹³Bi.

Certain isotopically-labelled peptide ligands of the invention, for example, those incorporating a radioactive isotope, are useful in drug and/or substrate tissue distribution studies. The peptide ligands of the invention can further have valuable diagnostic properties in that they can be used for detecting or identifying the formation of a complex between a labelled compound and other molecules, peptides, proteins, enzymes or receptors. The detecting or identifying methods can use compounds that are labelled with labelling agents such as radioisotopes, enzymes, fluorescent substances, luminous substances (for example, luminol, luminol derivatives, luciferin, aequorin and luciferase), etc. The radioactive isotopes tritium, i.e. ³H (T), and carbon-14, i.e. ¹⁴C, are particularly useful for this purpose in view of their ease of incorporation and ready means of detection.

Substitution with heavier isotopes such as deuterium, i.e. ²H (D), may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining target occupancy.

Isotopically-labeled compounds of peptide ligands of the invention can generally be prepared by conventional techniques known to those skilled in the art or by processes analogous to those described in the accompanying Examples using an appropriate isotopically-labeled reagent in place of the non-labeled reagent previously employed.

### Molecular Scaffold

In one embodiment, the molecular scaffold comprises a non-aromatic molecular scaffold. References herein to “non-aromatic molecular scaffold” refer to any molecular scaffold as defined herein which does not contain an aromatic (i.e. unsaturated) carbocyclic or heterocyclic ring system.

Suitable examples of non-aromatic molecular scaffolds are described in Heinis et al (2014) Angewandte Chemie, International Edition 53 (6) 1602-1606.

As noted in the foregoing documents, the molecular scaffold may be a small molecule, such as a small organic molecule.

In one embodiment the molecular scaffold may be a macromolecule. In one embodiment the molecular scaffold is a macromolecule composed of amino acids, nucleotides or carbohydrates.

In one embodiment the molecular scaffold comprises reactive groups that are capable of reacting with functional group(s) of the polypeptide to form covalent bonds.

The molecular scaffold may comprise chemical groups which form the linkage with a peptide, such as amines, thiols, alcohols, ketones, aldehydes, nitriles, carboxylic acids, esters, alkenes, alkynes, azides, anhydrides, succinimides, maleimides, alkyl halides and acyl halides.

An example of an αβ unsaturated carbonyl containing compound is 1,1′,1″-(1,3,5-triazinane-1,3,5-triyl)triprop-2-en-1-one (TATA) (Angewandte Chemie, International Edition (2014), 53 (6), 1602-1606).

### Synthesis

The peptides of the present invention may be manufactured synthetically by standard techniques followed by reaction with a molecular scaffold in vitro. When this is performed, standard chemistry may be used. This enables the rapid large scale preparation of soluble material for further downstream experiments or validation. Such methods could be accomplished using conventional chemistry such as that disclosed in Timmerman et al. (supra).

Thus, the invention also relates to manufacture of polypeptides selected as set out herein, wherein the manufacture comprises optional further steps as explained below. In one embodiment, these steps are carried out on the end product polypeptide made by chemical synthesis.

Peptides can also be extended, to incorporate for example another loop and therefore introduce multiple specificities.

To extend the peptide, it may simply be extended chemically at its N-terminus or C-terminus or within the loops using orthogonally protected lysines (and analogues) using standard solid phase or solution phase chemistry. Standard (bio)conjugation techniques may be used to introduce an activated or activatable N- or C-terminus. Alternatively, additions may be made by fragment condensation or native chemical ligation e.g. as described in (Dawson et al. 1994. Synthesis of Proteins by Native Chemical Ligation. Science 266:776-779), or by enzymes, for example using subtiligase as described in (Chang et al. Proc Natl Acad Sci USA. 1994 Dec. 20; 91 (26):12544-8 or in Hikari et al. Bioorganic & Medicinal Chemistry Letters Volume 18, Issue 22, 15 Nov. 2008, Pages 6000-6003).

Alternatively, the peptides may be extended or modified by further conjugation through disulphide bonds. This has the additional advantage of allowing the first and second peptide to dissociate from each other once within the reducing environment of the cell. In this case, the molecular scaffold (e.g. TATA) could be added during the chemical synthesis of the first peptide so as to react with the three cysteine groups; a further cysteine or thiol could then be appended to the N or C-terminus of the first peptide, so that this cysteine or thiol only reacted with a free cysteine or thiol of the second peptide, forming a disulfide-linked bicyclic peptide-peptide conjugate.

Similar techniques apply equally to the synthesis/coupling of two bicyclic and bispecific macrocycles, potentially creating a tetraspecific molecule.

Furthermore, addition of other functional groups or effector groups may be accomplished in the same manner, using appropriate chemistry, coupling at the N- or C-termini or via side chains. In one embodiment, the coupling is conducted in such a manner that it does not block the activity of either entity.

### Pharmaceutical Compositions

According to a further aspect of the invention, there is provided a pharmaceutical composition comprising a peptide ligand as defined herein in combination with one or more pharmaceutically acceptable excipients.

Generally, the present peptide ligands will be utilised in purified form together with pharmacologically appropriate excipients or carriers. Typically, these excipients or carriers include aqueous or alcoholic/aqueous solutions, emulsions or suspensions, including saline and/or buffered media. Parenteral vehicles include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition).

The compounds of the invention can be used alone or in combination with another agent or agents. The other agent for use in combination may be for example another antibiotic, or an antibiotic ‘adjuvant’ such as an agent for improving permeability into Gram-negative bacteria, an inhibitor of resistance determinants or an inhibitor of virulence mechanisms.

Suitable antibiotics for use in combination with the compounds of the invention include but are not limited to:

Beta lactams, such as penicillins, cephalosporins, carbapenems or monobactams. Suitable penicillins include oxacillin, methicillin, ampicillin, cloxacillin, carbenicillin, piperacillin, tricarcillin, flucloxacillin, and nafcillin; suitable cephalosporins include cefazolin, cefalexin, cefalothin, ceftazidime, cefepime, ceftobiprole, ceftaroline, ceftolozane and cefiderocol;

suitable carbapenems include meropenem, doripenem, imipenem, ertapenem, biapenem and tebipenem; suitable monobactams include aztreonam;

Lincosamides such as clindamycin and lincomycin;

Macrolides such as azithromycin, clarithromycin, erythromycin, telithromycin and solithromycin;

Tetracyclines such as tigecycline, omadacycline, eravacycline, doxycycline, and minocycline;

Quinolones such as ciprofloxacin, levofloxacin, moxifloxacin, and delafloxacin;

Rifamycins such as rifampicin, rifabutin, rifalazil, rifapentine, and rifaximin;

Aminoglycosides such as gentamycin, streptomycin, tobramycin, amikacin and plazomicin;

Glycopeptides such as vancomycin, teichoplanin, telavancin, dalbavancin, and oritavancin,

Pleuromutilins such as lefamulin

Oxazolidinones such as linezolid or tedizolid

Polymyxins such as polymyxin B or colistin;

Trimethoprim, iclaprim, sulfamethoxazole;

Metronidazole;

Fidaxomicin:

Mupirocin;

Fusidic acid;

Daptomycin;

Murepavidin;

Fosfomycin; and

Nitrofurantoin.

Suitable antibiotic ‘adjuvants’ include but are not limited to:

agents known to improve uptake into bacteria such as outer membrane permeabilisers or efflux pump inhibitors; outer membrane permeabilisers may include polymyxin B nonapeptide or other polymyxin analogues, or sodium edetate;

inhibitors of resistance mechanisms such as beta-lactamase inhibitors; suitable beta-lactamase inhibitors include clavulanic acid, tazobactam, sulbactam, avibactam, relebactam and nacubactam; and

inhibitors of virulence mechanisms such as toxins and secretion systems, including antibodies.

The compounds of the invention can also be used in combination with biological therapies such as nucleic acid based therapies, antibodies, bacteriophage or phage lysins.

The route of administration of pharmaceutical compositions according to the invention may be any of those commonly known to those of ordinary skill in the art. For therapy, the peptide ligands of the invention can be administered to any patient in accordance with standard techniques. Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eyedrops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly. Preferably, the pharmaceutical compositions according to the invention will be administered parenterally. The dosage and frequency of administration will depend on the age, sex and condition of the patient, concurrent administration of other drugs, counterindications and other parameters to be taken into account by the clinician.

The peptide ligands of this invention can be lyophilised for storage and reconstituted in a suitable carrier prior to use. This technique has been shown to be effective and art-known lyophilisation and reconstitution techniques can be employed. It will be appreciated by those skilled in the art that lyophilisation and reconstitution can lead to varying degrees of activity loss and that levels may have to be adjusted upward to compensate.

The compositions containing the present peptide ligands or a cocktail thereof can be administered for therapeutic treatments. In certain therapeutic applications, an adequate amount to accomplish at least partial inhibition, suppression, modulation, killing, or some other measurable parameter, of a population of selected cells is defined as a “therapeutically-effective dose”. Amounts needed to achieve this dosage will depend upon the severity of the disease and the general state of the patient's own immune system, but generally range from 10 μg to 250 mg of selected peptide ligand per kilogram of body weight, with doses of between 100 μg to 25 mg/kg/dose being more commonly used.

A composition containing a peptide ligand according to the present invention may be utilised in therapeutic settings to treat a microbial infection or to provide prophylaxis to a subject at risk of infection e.g. undergoing surgery, chemotherapy, artificial ventilation or other condition or planned intervention. In addition, the peptide ligands described herein may be used extracorporeally or in vitro selectively to kill, deplete or otherwise effectively remove a target cell population from a heterogeneous collection of cells. Blood from a mammal may be combined extracorporeally with the selected peptide ligands whereby the undesired cells are killed or otherwise removed from the blood for return to the mammal in accordance with standard techniques.

### Therapeutic Uses

The bicyclic peptides of the invention have specific utility as PBP3 or 3a binding agents.

Penicillin-binding proteins (PBPs) are a group of proteins that are characterized by their affinity for and binding of penicillin and they are present in many bacterial species. All 3-lactam antibiotics (except for tabtoxinine-β-lactam, which inhibits glutamine synthetase) bind to PBPs, which are essential for bacterial cell wall synthesis. PBPs are members of a subgroup of enzymes called transpeptidases. Specifically, some PBPs are DD-transpeptidases and bifunctional PBPs have transglycoylase activity. PBPs are all involved in the final stages of the synthesis of peptidoglycan, which is the major component of bacterial cell walls. Bacterial cell wall synthesis is essential to growth, cell division (thus reproduction) and maintaining the cellular structure in bacteria. Inhibition of PBPs leads to irregularities in cell wall structure such as elongation, lesions, loss of selective permeability, and eventual cell death and lysis. A review of PBPs is provided by Macheboeuf et al. (2006) FEMS Microbiology Reviews 30 (5), 673-691.

Thus, without being bound by theory it is believed that the peptide ligands of the present invention will be capable of causing bacterial growth inhibition, cell death and lysis by virtue of binding to PBPs and inhibiting cell wall synthesis. A review of PBPs as therapeutic targets is provided by Silver (2007) Nature Reviews Drug Discovery 6, 41-55 and Zervosen et al (2012) Molecules 17 (11), 12478-12505. It will be appreciated that the peptide ligands of the present invention may bind to the PBP at any site capable of interfering with the mechanism of action of said PBP. For example, the peptide ligand may bind to the active sites of said PBPs and inhibit the transpeptidase or transglycosylase. Alternatively, the peptide ligand may bind elsewhere on the PBP in order to interfere with its mechanism of action.

Polypeptide ligands selected according to the method of the present invention may be employed in in vivo therapeutic applications, in vitro and in vivo diagnostic applications, in vitro assay and reagent applications, and the like. In some applications, such as vaccine applications, the ability to elicit an immune response to predetermined ranges of antigens can be exploited to tailor a vaccine to specific diseases and pathogens.

Substantially pure peptide ligands of at least 90 to 95% homogeneity are preferred for administration to a mammal, and 98 to 99% or more homogeneity is most preferred for pharmaceutical uses, especially when the mammal is a human. Once purified, partially or to homogeneity as desired, the selected polypeptides may be used diagnostically or therapeutically (including extracorporeally) or in developing and performing assay procedures, immunofluorescent stainings and the like (Lefkovite and Pernis, (1979 and 1981) Immunological Methods, Volumes I and II, Academic Press, NY).

According to a further aspect of the invention, there is provided a peptide ligand as defined herein, for use in suppressing or treating a disease or disorder mediated by bacterial infection or for providing prophylaxis to a subject at risk of infection.

According to a further aspect of the invention, there is provided a method of suppressing or treating a disease or disorder mediated by bacterial infection or for providing prophylaxis to a subject at risk of infection, which comprises administering to a patient in need thereof the peptide ligand as defined herein.

The peptide ligands of the invention or pharmaceutical compositions comprising said peptide ligands are useful for the treatment of skin and soft tissue infections, gastrointestinal infection, urinary tract infection, pneumonia, sepsis, intra-abdominal infection and obstetrical/gynaecological infections. The infections may be caused by Gram-positive bacteria, such as S. pneumoniae, or Gram-negative bacteria, such as E. coli, P. aeruginosa and A. baumannii, or may be due to more than one species of bacterium.

In one embodiment, the disease or disorder mediated by bacterial infection is selected from:
pertussis (which may be caused by Bordetella pertussis);
tetanus (which may be caused by Clostrium tetani);
diphtheria (which may be caused by Corynebacterium diphtheriae);
echinococcal disease (which may be caused by Echinococcus); diarrhea, hemolytic uremic syndrome or urinary tract infection (which may be caused by Escherichia coli);
respiratory infections or meningitis (which may be caused by Haemophilus influenzae);
gastritis, peptic ulcer disease or gastric neoplasms (which may be caused by Helicobacter pylori);
tuberculosis (which may be caused by Mycobacterium tuberculosis);
meningitis, pneumonia, bacteremia or otitis media (which may be caused by Pneumococcus);
food poisoning (which may be caused by Salmonella);
shigellosis or gastroenteritis (which may be caused by Shigella); and
cholera (which may be caused by Vibrio cholerae).

References herein to the term “suppression” refers to administration of the composition after an inductive event, but prior to the clinical appearance of the disease. “Treatment” involves administration of the protective composition after disease symptoms become manifest.

Animal model systems which can be used to screen the effectiveness of the peptide ligands in protecting against or treating the disease are available.

The invention is further described below with reference to the following examples.

### EXAMPLES

### Materials and Methods

### Peptide Synthesis

Peptide synthesis was based on Fmoc chemistry, using a Symphony peptide synthesiser manufactured by Peptide Instruments and a Syro II synthesiser by MultiSynTech. Standard Fmoc-amino acids were employed (Sigma, Merck), with appropriate side chain protecting groups: where applicable standard coupling conditions were used in each case, followed by deprotection using standard methodology.

Alternatively, peptides were purified using HPLC and following isolation they were modified with 1,3,5-Triacryloylhexahydro-1,3,5-triazine (TATA, Sigma). For this, linear peptide was diluted with 50:50 MeCN:H₂O up to ˜35 mL, ˜500 μL of 100 mM TATA in acetonitrile was added, and the reaction was initiated with 5 mL of 1 M NH₄HCO₃ in H₂O. The reaction was allowed to proceed for ˜30-60 min at RT, and lyophilised once the reaction had completed (judged by MALDI). Once completed, 1 ml of 1M L-cysteine hydrochloride monohydrate (Sigma) in H₂O was added to the reaction for ˜60 min at RT to quench any excess TATA.

Following lyophilisation, the modified peptide was purified as above, while replacing the Luna C8 with a Gemini C18 column (Phenomenex), and changing the acid to 0.1% trifluoroacetic acid. Pure fractions containing the correct TATA-modified material were pooled, lyophilised and kept at −20° C. for storage.

All amino acids, unless noted otherwise, were used in the L-configurations.

In some cases peptides are converted to activated disulfides prior to coupling with the free thiol group of a toxin using the following method; a solution of 4-methyl(succinimidyl 4-(2-pyridylthio)pentanoate) (100 mM) in dry DMSO (1.25 mol equiv) was added to a solution of peptide (20 mM) in dry DMSO (1 mol equiv). The reaction was well mixed and DIPEA (20 mol equiv) was added. The reaction was monitored by LC/MS until complete.

### Biological Data

### Fluorescence Polarisation Direct Binding Assay

Fluorescence polarisation was carried out using fluorescein-labelled peptides with unmodified PBP protein and measured using a PHERAstar FS by BMG Labtech fitted with a FP 485 520 520 optic module.

Fluorescent peptides at 10 mM in DMSO were diluted to 2.5 nM in binding buffer (10 mM HEPES, pH8, 300 mM NaCl, 2% glycerol). A two-fold dilution series of PBP protein was then prepared across 12 wells in binding buffer, with the highest concentration being 21 μM, and the lowest concentration being 17 nM.

10 μl diluted fluorescent peptide (2.5 nM) was added into 12 wells of a 384-Well NBS™ Low Volume Microplate (Fisher Scientific). 10 μl PBP dilution series was then added to the wells containing the fluorescent peptide, and 5 μl binding buffer was added to bring the total volume to 25 μl and the final concentration of peptide tracer to 1 nM. A control well lacking PBP protein was prepared with a final peptide concentration of 1 nM to a final volume of 25 μl in binding buffer. Fluorescence polarisation was measured every 5 minutes for a period of one hour at room temperature. The gain and focal height were optimised using the control well lacking protein. Wells were excited at 485 nm, and emission detection was set at 520 nm.

Data were analysed in GraphPad software to derive values for the dissociation constant. Experiments were repeated at least three times.

Certain peptide ligands of the invention were tested in the above mentioned binding assay and the results are shown in Table 1 for binding to the indicated PBP:

TABLE 1
Direct Binding Data for Selected Peptide Ligands of the Invention

| Peptide Number | Kd (μM) | PBP Tested |
|---|---|---|
| BCY12806 | 2.10 | 3 (E. coli) |
| BCY12820 | 0.005 | 3 (E. coli) |

A control inhibitor, Bocillin, bound to PBP3 of E. coli with a Kd of 0.44 μM. Thus, this data demonstrates that the peptide ligands of the present invention selectively bind PBPs with high affinity. Of note, the direct binding values presented herein utilise a peptide ligand bound to a fluorescence tracer molecule.

### Fluorescence Polarisation Competition Binding Assay

Fluorescence polarisation competition was carried out using a BODIPY labelled Penicillin tracer and unlabelled peptides, for competition to an unmodified PBP protein. Polarisation was measured using a PHERAstar FS by BMG Labtech fitted with a FP 485 520 520 optic module.

Fluorescent BODIPY labelled penicillin at 5 mM in DMSO were diluted to 6.25 nM in binding buffer (10 mM HEPES, pH8, 300 mM NaCl, 2% glycerol). Unmodified PBP were diluted to 2 μM in binding buffer. A two-fold dilution series of unmodified peptide was prepared across 12 wells in binding buffer, with the highest final well concentration being 60 μM, and the lowest concentration being 50 nM. 5 μl of the unmodified peptide dilution series or Carbenicillin were added to 12 wells of a 384-Well NBS™ Low Volume Microplate (Fisher Scientific). 10 μl diluted fluorescent BODIPY labelled penicillin (6.25 nM) were then added to the 12 wells containing the unmodified peptide dilutions. 10 μl of unmodified PBP (2 μM) were then added to the 12 wells containing unmodified peptide and fluorescent BODIPY labelled penicillin to bring the total volume to 25 μl, and the final concentration of Fluorescent BODIPY labelled penicillin to 2.5 nM and unmodified PBP to 800 nM.

A control well lacking unmodified peptide was prepared with a final fluorescent BODIPY labelled penicillin concentration of 2.5 nM and a final concentration of unmodified PBP of 800 nM to a final volume of 25 μl in binding buffer. A second control well lacking unmodified peptide and unmodified PBP was prepared with a final fluorescent BODIPY labelled penicillin concentration of 2.5 nM to a final volume of 25 μl in binding buffer.

Fluorescence polarisation was measured every 5 minutes for a period of one hour at room temperature. The gain and focal height were optimised using the control well lacking unmodified peptide and unmodified PBP. Wells were excited at 485 nm, and emission detection was set at 520 nm.

Data were analysed in GraphPad software to derive values for the inhibition constant. Experiments were repeated at least three times.

Certain peptide ligands of the invention were tested in the above mentioned competition assay and the results are shown in Table 2:

TABLE 2
Competition Binding Data for Selected
Peptide Ligands of the Invention

| Peptide Number | Ki (μM) |
|---|---|
| BCY12123 | 1.39 |

As can be seen from the data presented herein, the tested peptide ligand of the present invention selectively binds to and inhibit the beta-lactam binding of PBPs.

A further fluorescence polarisation competition study was carried out using BCY12820 as a tracer and the results are shown in Table 3:

TABLE 3
Competition Binding Data for Selected
Peptide Ligands of the Invention

| Peptide Number | Ki (μM) |
|---|---|
| BCY12820 | 0.005 |
| BCY12742 | 0.003 |
| BCY13226 | 0.020 |
| BCY13227 | 0.027 |
| BCY13419 | 0.040 |
| BCY13420 | 0.029 |
| BCY13421 | 0.035 |
| BCY13422 | 0.043 |
| BCY13424 | 0.025 |
| BCY13656 | 0.005 |
| BCY13657 | 0.331 |
| BCY13658 | 0.072 |
| BCY13659 | 0.004 |
| BCY13660 | 0.294 |
| BCY13663 | 0.012 |
| BCY13664 | 0.030 |
| BCY13747 | 11.277 |
| BCY13748 | 10.517 |
| BCY13749 | 5.560 |
| BCY13750 | 10.899 |
| BCY13751 | 9.644 |
| BCY13753 | 21.672 |
| BCY13754 | 12.564 |
| BCY13755 | 14.459 |
| BCY13756 | 13.997 |
| BCY13757 | 14.838 |
| BCY13758 | 15.247 |
| BCY13759 | 11.443 |
| BCY13760 | 8.947 |
| BCY13761 | 12.635 |
| BCY13762 | 12.983 |
| BCY13763 | 9.820 |
| BCY13764 | <4.08 |
| BCY13765 | 1.470 |
| BCY13766 | 4.680 |
| BCY13767 | 5.390 |
| BCY13768 | <3.82 |
| BCY13769 | 4.180 |
| BCY13949 | 45.379 |
| BCY13952 | 118.089 |
| BCY13953 | 38.418 |
| BCY13961 | 13.213 |
| BCY13962 | 12.053 |
| BCY13963 | 12.128 |
| BCY13964 | 11.415 |
| BCY13965 | 11.230 |
| BCY13966 | 10.452 |
| BCY13967 | 13.973 |
| BCY13968 | 9.473 |
| BCY13969 | 7.317 |
| BCY13970 | 7.867 |
| BCY13971 | 12.118 |
| BCY13972 | 10.649 |
| BCY13973 | 10.910 |
| BCY13975 | 7.900 |
| BCY13976 | 7.350 |
| BCY13977 | 5.018 |
| BCY13978 | 14.821 |
| BCY13979 | 8.464 |
| BCY13980 | 7.990 |
| BCY13981 | 2.133 |

### Minimum Inhibition Concentration and Minimum Bactericidal Concentration Assays

Minimum inhibitory concentration (MIC) assays were carried out using E. coli strains from the Zgurskaya lab engineered to harbour an inducible pore to make the outer membrane permeable (Krishnamoorthy et al. (2016) doi: https://doi.org/10.1128/AAC.01882-16). Strains used: GKCW101; GKCW102; GKCW103; and GKCW104.

Overnight cultures of bacteria were first prepared by transferring a single bacterial colony into 5 mL cation-adjusted Mueller-Hinton broth (CA-MHB) supplemented with kanamycin at 50 μg/mL. The following day, overnight cultures were diluted 1/100 in 25 mL CA-MHB with 50 μg/mL kanamycin and cultured until the optical density reached 0.3 as measured on a spectrometer at 600 nm.

Filter-sterilised arabinose was then added to a concentration of 0.1% w/v and subsequently cultured until the optical density at 600 nm equalled to 1.

The cultured medium was then diluted to 1×10⁶ CFU mL⁻¹ in CA-MHB supplemented with 0.1% w/v arabinose and 50 μg/mL kanamycin.

In a 96 well microtitre plate 100 μL CA-MHB was dispensed into wells in columns 2-12, with 200 μL being added into the wells of column 1 to prepare a 2-fold serial dilution.

A maximum 8 μl of peptide ligand was then added into the wells of column 1 and diluted two-fold across the plate. Positive controls (effective antibiotic) and DMSO controls were included in rows G and H respectively. Plates were then sealed with a gas-permeable seal and incubated at 37° C. for 18 hours. The optical density at 600 nm was then measured for each of the wells using a PheraStar FSx plate reader. MIC values were determined as the cutoff concentration between visible growth and no growth of the bacteria.

Minimal bactericidal concentrations (MBC) were determined by dispensing 5 μL of MIC culture from each well onto a large LB agar plate (100 mL), followed by overnight incubation at 37° C. The MBC was calculated as the concentration of antibiotic at which no colonies were detected on the agar.

Alternatively, MIC of peptide ligands were evaluated using a 10 μl MIC assay carried out using E. coli (ATCC 25922), S. typhimurium (ATCC 19585) and E. cloacae (NCTC 13405). Test compounds were dissolved at high concentration in the required solvent. Where DMSO was used to dissolve test compounds, a DMSO control with equivalent concentrations of DMSO was included for each test organism. For each test organism, a control antibiotic (for which susceptibility of the test organism was known) was tested in parallel. This allowed comparison of results between runs and validated the testing procedure and organism.

Using aseptic technique, serial 2-fold dilutions (across a 10 point range) of the test compounds were made in 5 μl of cation-adjusted Muller Hinton broth (CA-MHB), on the inverted lid of a 96-well plate. Inoculum for each test organism was made in phosphate-buffered saline (PBS) to match the turbidity of a 0.5 McFarland standard, and then diluted 100-fold. Each well (except negative control well) was inoculated with 5 μl of inoculum. For each test antibiotic, a negative control (no bacteria) was included, consisting of CA-MHB only; along with a positive control (no antibiotic) with CA-MHB and bacteria only. The base of the 96 well plate was used as a lid, and the plate was incubated inside a humidor to reduce evaporation. Humidors were constructed by sealing the test plate within a pre-warmed plastic box, containing tissue saturated with PBS. Test plates were incubated at the temperature and time period dictated by CLSI guidelines for MIC determination. MIC was evaluated based on turbidity, using white light shone through the base of the plate to illuminate droplets.

Peptides of the invention were analysed for their inhibition specifically of E. coli GKCW102 (WT-pore) and the results are shown in Table 4:

TABLE 4
MIC Data for Selected Peptide Ligands of the Invention

| | MIC (E. coli |
|---|---|
| | GKCW102) |
| Peptide Number | (μM) |
| BCY12742 | 0.195 |
| BCY13226 | 0.700 |
| BCY13227 | 1.000 |
| BCY13656 | 0.727 |
| BCY13657 | 114.000 |
| BCY13658 | 4.500 |
| BCY13659 | 0.355 |
| BCY13660 | 40.000 |
| BCY13663 | 0.258 |
| BCY13664 | 0.797 |
| BCY13747 | 0.080 |
| BCY13748 | 0.170 |
| BCY13749 | 0.145 |
| BCY13750 | 0.363 |
| BCY13751 | 0.084 |
| BCY13753 | 0.313 |
| BCY13754 | 0.133 |
| BCY13755 | 0.184 |
| BCY13756 | 0.101 |
| BCY13757 | 0.239 |
| BCY13758 | 0.148 |
| BCY13759 | 0.670 |
| BCY13760 | 0.156 |
| BCY13761 | 0.168 |
| BCY13762 | 0.166 |
| BCY13763 | 1.484 |
| BCY13764 | 0.184 |
| BCY13765 | 0.154 |
| BCY13766 | 0.053 |
| BCY13767 | 0.166 |
| BCY13768 | 0.297 |
| BCY13769 | 0.281 |
| BCY13961 | 0.730 |
| BCY13962 | 0.150 |
| BCY13963 | 0.150 |
| BCY13964 | 0.190 |
| BCY13965 | 0.620 |
| BCY13966 | 0.040 |
| BCY13967 | 0.330 |
| BCY13968 | 0.340 |
| BCY13969 | 1.220 |
| BCY13970 | 0.680 |
| BCY13971 | 1.270 |
| BCY13972 | 0.170 |
| BCY13973 | 2.780 |
| BCY13975 | 0.080 |
| BCY13976 | 0.310 |
| BCY13977 | 0.360 |
| BCY13978 | 0.660 |
| BCY13979 | 0.080 |
| BCY13980 | 0.320 |
| BCY13981 | 0.040 |

These data demonstrate the ability of peptide ligands of the present invention to inhibit bacterial growth in E. coli.

1. A peptide ligand capable of binding to penicillin-binding protein 3 (PBP3) comprising a polypeptide which comprises at least three cysteine residues, separated by at least two loop sequences, and a molecular scaffold which forms covalent bonds with the cysteine residues of the polypeptide such that at least two polypeptide loops are formed on the molecular scaffold.

2. The peptide ligand as defined in claim 1, wherein said loop sequences comprise 3, 4, 5 or 9 amino acids.

3. The peptide ligand as defined in claim 1 or claim 2, wherein the PBP3 is a PBP3 which is present within one or more pathogenic bacterial species.

4. The peptide ligand as defined in claim 3, wherein the one or more pathogenic bacterial species is selected from any of: Acinetobacter baumannii, Bacillus anthracis, Bordetella pertussis, Borrelia burgdorferi, Brucella abortus, Brucella canis, Brucella melitensis, Brucella suis, Campylobacter jejuni, Chlamydia pneumonia, Chlamydia trachomatis, Chlamydophila psittaci, Clostridium botulinum, Clostridium difficile, Clostridium perfringens, Clostrium tetani, Corynebacterium diphtheriae, Echinococcus, Enterococcus faecalis, Enterococcus faecium, Escherichia coli (such as Enterotoxigenic E. coli, Enteropathogenic E. coli, Enterohemorragic E. coli or Enteroaggregative E. coli), Francisella tularensis, Haemophilus influenzae, Helicobacter pylori, Klebsiella pneumoniae, Legionella pneumophila, Leptospira interrogans, Listeria monocytogenes, Mycobacterium leprae, Mycobacterium tuberculosis, Mycobacterium ulcerans, Mycoplasma pneumonia, Neisseria gonorrhoeae, Neisseria meningitides, Pneumococcus, Pseudomonas aeruginosa, Rickettsia rickettsia, Salmonella such as, Salmonella bongori, Salmonella enterica, Salmonella subterranean, Salmonella typhi or Salmonella typhimurium, Shigella (such as Shigella sonnei or Shigella dysenteriae), Staphylococcus aureus (such as MRSA), Staphylococcus epidermidis, Staphylococcus saprophyticus, Streptococcus agalactiae, Streptococcus pneumoniae, Streptococcus pyogenes, Treponema pallidum, Vibrio cholerae or Yersinia pestis.

5. The peptide ligand as defined in any one of claims 1 to 4, wherein the PBP3 is E. coli PBP3 and the peptide ligand comprises an amino acid sequence selected from:
(SEQ ID NO: 1)
CᵢSFPKCᵢᵢPWVEGCᵢᵢᵢ
(herein referred to as BCY13949);
(SEQ ID NO: 2)
CᵢRTFGCᵢᵢWWEGCᵢᵢᵢ;
(SEQ ID NO: 3)
CᵢLENCᵢᵢYYVPYYGYACᵢᵢᵢ;
(SEQ ID NO: 4)
CᵢSFP[K(Ac)]CᵢᵢPWVEGCᵢᵢᵢ;
(SEQ ID NO: 5)
CᵢYFPGCᵢᵢPWVENCᵢᵢᵢ;
(SEQ ID NO: 6)
CᵢQFPVCᵢᵢPWVEGCᵢᵢᵢ;
(SEQ ID NO: 7)
CᵢSFPMCᵢᵢPWVENCᵢᵢᵢ;
(SEQ ID NO: 8)
CᵢSFPKCᵢᵢPWVE[dK]Cᵢᵢᵢ;
(SEQ ID NO: 9)
CᵢSFPKCᵢᵢPWVE[dA]Cᵢᵢᵢ;
(SEQ ID NO: 10)
CᵢSFPKCᵢᵢP[2Nal]VEGCᵢᵢᵢ;
(SEQ ID NO: 11)
CᵢSFPKCᵢᵢP[1Nal]VEGCᵢᵢᵢ;
(SEQ ID NO: 12)
CᵢSFP[HArg]CᵢᵢPWVEGCᵢᵢᵢ;
(SEQ ID NO: 13)
CᵢAFPKCᵢᵢPWVEGCᵢᵢᵢ;
(SEQ ID NO: 14)
CᵢSAPKCᵢᵢPWVEGCᵢᵢᵢ;
(SEQ ID NO: 15)
CᵢSFAKCᵢᵢPWVEGCᵢᵢᵢ;
(SEQ ID NO: 16)
CᵢSFPACᵢᵢPWVEGCᵢᵢᵢ;
(SEQ ID NO: 17)
CᵢSFPKCᵢᵢAWVEGCᵢᵢᵢ;
(SEQ ID NO: 18)
CᵢSFPKCᵢᵢPWVAGCᵢᵢᵢ;
(SEQ ID NO: 19)
CᵢSFPKCᵢᵢPWVEACᵢᵢᵢ;
(SEQ ID NO: 20)
CᵢKFPVCᵢᵢPWVEYCᵢᵢᵢ;
(SEQ ID NO: 21)
CᵢHFPTCᵢᵢPWVEWCᵢᵢᵢ;
(SEQ ID NO: 22)
CᵢTFPACᵢᵢPWVEYCᵢᵢᵢ;
(SEQ ID NO: 23)
CᵢLFPGCᵢᵢPWVEHCᵢᵢᵢ;
(SEQ ID NO: 24)
CᵢFFPQCᵢᵢPWVEGCᵢᵢᵢ;
(SEQ ID NO: 25)
CᵢHFPVCᵢᵢPWVENCᵢᵢᵢ;
(SEQ ID NO: 26)
CᵢHFPKCᵢᵢPWVEGCᵢᵢᵢ;
(SEQ ID NO: 27)
CᵢEFPKCᵢᵢPWVEGCᵢᵢᵢ;
(SEQ ID NO: 28)
CᵢYFPKCᵢᵢPWVEGCᵢᵢᵢ;
(SEQ ID NO: 29)
CᵢSYPKCᵢᵢPWVEGCᵢᵢᵢ;
(SEQ ID NO: 30)
CᵢSFPKCᵢᵢPWVEHCᵢᵢᵢ;
(SEQ ID NO: 31)
CᵢTFPKCᵢᵢPWVEGCᵢᵢᵢ;
(SEQ ID NO: 32)
CᵢSFPKCᵢᵢPWVENCᵢᵢᵢ;
(SEQ ID NO: 33)
CᵢWFPNCᵢᵢPWVEGCᵢᵢᵢ;
(SEQ ID NO: 34)
CᵢYYPKCᵢᵢPWVEGCᵢᵢᵢ;
(SEQ ID NO: 35)
CᵢRFPKCᵢᵢPWVEGCᵢᵢᵢ:
(SEQ ID NO: 36)
CᵢSFPICᵢᵢPWVEGCᵢᵢᵢ;
(SEQ ID NO: 37)
CᵢVYPKCᵢᵢPWVEGCᵢᵢᵢ;
(SEQ ID NO: 38)
CᵢIYPKCᵢᵢPWVEGCᵢᵢᵢ;
(SEQ ID NO: 39)
CᵢSF[dA]KCᵢᵢPWVEGCᵢᵢᵢ;
(SEQ ID NO: 40)
CᵢSFP[dA]CᵢᵢPWVEGCᵢᵢᵢ;
(SEQ ID NO: 41)
CᵢSFPTCᵢᵢPWVEGCᵢᵢᵢ;
(SEQ ID NO: 42)
CᵢVFPKCᵢᵢPWVEGCᵢᵢᵢ;
(SEQ ID NO: 43)
CᵢSFPHCᵢᵢPWVEGCᵢᵢᵢ;
(SEQ ID NO: 44)
CᵢHYPKCᵢᵢPWVEGCᵢᵢᵢ;
(SEQ ID NO: 45)
CᵢEYPKCᵢᵢPWVEGCᵢᵢᵢ;
(SEQ ID NO: 46)
CᵢSFPLCᵢᵢPWVEGCᵢᵢᵢ;
(SEQ ID NO: 47)
CᵢAFPHCᵢᵢPWVEFCᵢᵢᵢ;
(SEQ ID NO: 48)
CᵢQFPGCᵢᵢPWVEFCᵢᵢᵢ;
(SEQ ID NO: 49)
CᵢIFPKCᵢᵢPWVEGCᵢᵢᵢ;
(SEQ ID NO: 50)
CᵢSFPSCᵢᵢPWVEHCᵢᵢᵢ;
(SEQ ID NO: 51)
CᵢWFPGCᵢᵢPWVEHCᵢᵢᵢ;
(SEQ ID NO: 52)
CᵢRFPDCᵢᵢPWVEHCᵢᵢᵢ;
(SEQ ID NO: 53)
CᵢHFPTCᵢᵢPWVEYCᵢᵢᵢ;
(SEQ ID NO: 54)
CᵢVFPACᵢᵢPWVEYCᵢᵢᵢ;
(SEQ ID NO: 55)
CᵢNFPECᵢᵢPWVEHCᵢᵢᵢ;
(SEQ ID NO: 56)
CᵢRFPNCᵢᵢPWVEHCᵢᵢᵢ;
and
(SEQ ID NO: 57)
CᵢVFPTCᵢᵢPWVEHCᵢᵢᵢ;
wherein HArg represent homoarginine, 1Nal represents 1-naphthylalanine, 2Nal represents 2-naphthylalanine and Cᵢ, Cᵢᵢ and Cᵢᵢᵢ represent first, second and third cysteine residues, respectively, or a pharmaceutically acceptable salt thereof, such as:
Ac-(SEQ ID NO: 1)
(herein referred to as BCY12742);
A-(SEQ ID NO: 1)-A-[Sar₆]-[KFI]
(herein referred to as BCY12806);
[FI]-G-[Sar₅]-A-(SEQ ID NO: 1)-A
(herein referred to as BCY12820);
SGH-(SEQ ID NO: 1)-A
(herein referred to as BCY13761);
Ac-(SEQ ID NO: 2)
(herein referred to as BCY12744);
[FI]-G-[Sar₅]-A-(SEQ ID NO: 2)-A
(herein referred to as BCY12822);
A-(SEQ ID NO: 2)-A-[Sar₆]-[KFI]
(herein referred to as BCY12825);
A-(SEQ ID NO: 3)-A
(herein referred to as BCY12123);
Ac-(SEQ ID NO: 4)
(herein referred to as BCY12834);
A-(SEQ ID NO: 5)-A
(herein referred to as BCY13981);
A-(SEQ ID NO: 6)-A
(herein referred to as BCY13226);
A-(SEQ ID NO: 7)-A
(herein referred to as BCY13227);
A-(SEQ ID NO: 8)-A
(herein referred to as BCY13419);
A-(SEQ ID NO: 9)-A
(herein referred to as BCY13420);
A-(SEQ ID NO: 10)-A
(herein referred to as BCY13421);
A-(SEQ ID NO: 11)-A
(herein referred to as BCY13422);
A-(SEQ ID NO: 12)-A
(herein referred to as BCY13424);
A-(SEQ ID NO: 13)-A
(herein referred to as BCY13656);
A-(SEQ ID NO: 14)-A
(herein referred to as BCY13657);
A-(SEQ ID NO: 15)-A
(herein referred to as BCY13658);
A-(SEQ ID NO: 16)-A
(herein referred to as BCY13659);
A-(SEQ ID NO: 17)-A
(herein referred to as BCY13660);
A-(SEQ ID NO: 18)-A
(herein referred to as BCY13663);
A-(SEQ ID NO: 19)-A
(herein referred to as BCY13664);
A-(SEQ ID NO: 20)-A
(herein referred to as BCY13747);
A-(SEQ ID NO: 21)-A
(herein referred to as BCY13748);
A-(SEQ ID NO: 22)-A
(herein referred to as BCY13749);
A-(SEQ ID NO: 23)-A
(herein referred to as BCY13750);
A-(SEQ ID NO: 24)-A
(herein referred to as BCY13751);
A-(SEQ ID NO: 25)-A
(herein referred to as BCY13753);
A-(SEQ ID NO: 26)-A
(herein referred to as BCY13754);
EDR-(SEQ ID NO: 26)-A
(herein referred to as BCY13760);
A-(SEQ ID NO: 27)-A
(herein referred to as BCY13755);
A-(SEQ ID NO: 28)-A
(herein referred to as BCY13756);
A-(SEQ ID NO: 29)-A
(herein referred to as BCY13757);
A-(SEQ ID NO: 30)-A
(herein referred to as BCY13758);
A-(SEQ ID NO: 30)-SSI
(herein referred to as BCY13762);
ESW-(SEQ ID NO: 31)-A
(herein referred to as BCY13759);
A-(SEQ ID NO: 31)-A
(herein referred to as BCY13963);
A-(SEQ ID NO: 32)-KVD
(herein referred to as BCY13763);
A-(SEQ ID NO: 33)-A
(herein referred to as BCY13764);
A-(SEQ ID NO: 34)-A
(herein referred to as BCY13765);
A-(SEQ ID NO: 35)-A
(herein referred to as BCY13766);
A-(SEQ ID NO: 36)-A
(herein referred to as BCY13767);
A-(SEQ ID NO: 37)-A
(herein referred to as BCY13768);
A-(SEQ ID NO: 38)-A
(herein referred to as BCY13769);
A-(SEQ ID NO: 39)-A
(herein referred to as BCY13952);
A-(SEQ ID NO: 40)-A
(herein referred to as BCY13953);
A-(SEQ ID NO: 41)-A
(herein referred to as BCY13961);
A-(SEQ ID NO: 42)-A
(herein referred to as BCY13962);
A-(SEQ ID NO: 43)-A
(herein referred to as BCY13965);
A-(SEQ ID NO: 44)-A
(herein referred to as BCY13966);
A-(SEQ ID NO: 45)-A
(herein referred to as BCY13967);
A-(SEQ ID NO: 46)-A
(herein referred to as BCY13968);
A-(SEQ ID NO: 47)-A
(herein referred to as BCY13969);
A-(SEQ ID NO: 48)-A
(herein referred to as BCY13970);
A-(SEQ ID NO: 49)-A
(herein referred to as BCY13971);
A-(SEQ ID NO: 50)-A
(herein referred to as BCY13972);
A-(SEQ ID NO: 51)-A
(herein referred to as BCY13973);
A-(SEQ ID NO: 52)-A
(herein referred to as BCY13975);
A-(SEQ ID NO: 53)-A
(herein referred to as BCY13976);
A-(SEQ ID NO: 54)-A
(herein referred to as BCY13977);
A-(SEQ ID NO: 55)-A
(herein referred to as BCY13978);
A-(SEQ ID NO: 56)-A
(herein referred to as BCY13979);
and
A-(SEQ ID NO: 57)-A
(herein referred to as BCY13980).

6. The peptide ligand as defined in any one of claims 1 to 5, wherein the molecular scaffold is 1,1′,1″-(1,3,5-triazinane-1,3,5-triyl)triprop-2-en-1-one (TATA).

7. The peptide ligand as defined in any one of claims 1 to 6, wherein the pharmaceutically acceptable salt is selected from the free acid or the sodium, potassium, calcium and ammonium salt.

8. A pharmaceutical composition which comprises the peptide ligand of any one of claims 1 to 7, in combination with one or more pharmaceutically acceptable excipients.

9. The pharmaceutical composition as defined in claim 8, which additionally comprises one or more therapeutic agents.

10. The peptide ligand as defined in any one of claims 1 to 7 or the pharmaceutical composition as defined in claim 8 or claim 9, for use in suppressing or treating a disease or disorder mediated by bacterial infection or for providing prophylaxis to a subject at risk of infection.
